(19) United States
(12) Patent Application Publication
(10) Pub. No.: US 2008/0032390 A1
(43) Pub. Date: Feb. 7, 2008

(54) Method and Device for Control of Diffusive Transport

(76) Inventors: Ivar Meyvantsson, Madison, WI (US); David J. Beebe, Monona, WI (US)

Correspondence Address: BOYLE FREDRICKSON S.C., 840 North Plankinton Avenue, MILWAUKEE, WI, 53203, US

(21) Appl. No.: 11/462,585

(22) Filed: Aug. 4, 2006

(51) Int. Cl. C12M 1/40

(52) U.S. Cl. 435/286.5; 422/102; 435/287.1; 435/288.3

(57) ABSTRACT

A method and apparatus are provided for regulating diffusive transport of particles between first and second portions of a channel network in a microfluidic device. The first and second portions of the channel network are in fluid communication. A first object is deposited in a first portion of the channel network and a second object is deposited in the second portion of the channel network. The diffusive transport of particles between the first and second portions of the channel network is controlled so as to allow for the study of reciprocal signaling between the objects.

### FIELD OF THE INVENTION

This invention relates generally to microfluidic devices, and in particular, to a method and device for effectuating dynamic control of diffusive transport that occurs between selected portions of a channel network of a microfluidic device.

### BACKGROUND AND SUMMARY OF THE INVENTION

As is known, cells do not live in isolation. In all multi-cellular organisms, such as the human body, the cells within the body continually receive and send signals that coordinate the growth, differentiation, and metabolism of the cells in diverse tissues and organs. For example, morphogens are signaling molecules secreted by cells. In embryos, concentration gradients of morphogens play a key role in the formation and differentiation of many tissues, as well as, set the stage for the formation of organs. Further, it has been found that more intricate structures are formed by local, and sometimes reciprocal, interactions between different cell types. For example, the hair follicle is formed and maintained according to reciprocal signaling between the epidermal and dermal components of the skin. Reciprocal interactions also take place in the nervous system during formation of axon scaffolds that are precursors to neuronal connections, as well as, in regeneration wherein glial signals can, in fact, be detrimental to the repair process. As such, it can be appreciated that a better understanding of tissue level signaling is important for the development of new therapies and for tissue engineering. In addition, robust tools for in vitro modeling may have utility for the discovery of new drugs that target signaling pathways.

To study reciprocal signaling in vitro, one can employ cells that either over-express a component of a pathway or have dominant negative allele. However, this process requires the prior knowledge (or at least a hint) of the pathways involved. Also, genetic manipulations are difficult if the interaction between the cells involves multiple pathways. Pharmacological inhibitors could be used, but these inhibitors are only available for some signaling cascades and tend to lack specificity.

An alternative way of studying reciprocal signaling is to observe two or more cell types involved as they are joined in co-culture or separated after having been in contact. Traditional co-culture techniques do not enable easy cessation of cell to cell communication within a co-culture. In a mixed co-culture, it is not possible to remove all signals originating with one cell type, while leaving the second cell type unaffected. For example, when using filter well inserts, cells are usually seeded on either side of a membrane. It can be appreciated that any effort to remove one cell type from a well is likely to disturb the other cell type. Even if one cell type is seeded on the bottom of a well and the other on a filter insert, it will be difficult and time consuming to remove the filter without causing crosstalk between the wells.

Therefore, it is a primary object and feature of the present invention to provide a method and a device for studying reciprocal signaling between two or more cells positioned within a channel network of a microfluidic device.

It is a further object and feature of the present invention to provide a method and a device for studying reciprocal signaling between two or more cells positioned within a channel network of a microfluidic device that allows for dynamic control of diffusive transport that occurs between the cells.

It is a still further object and feature of the present invention to provide a method and a device for studying reciprocal signaling between two or more cells positioned within a channel network of a microfluidic device that allows for the easy cessation of cell to cell communication.

In accordance with the present invention, a method is provided of controlling diffusive transport between first and second portions of a channel network in a microfluidic device. The first and second portions of the channel network are in fluid communication. The method includes the step of providing a flow path in the microfluidic device. The flow path has an input and an output and extends between the first and second portions of the channel network. A predetermined fluid flows along the flow path at a flow rate so as to selectively control diffusive transport of particle between the first and second portions of the channel network.

The step of flowing the predetermined fluid includes the additional step of increasing the flow rate of the predetermined fluid to predetermined level to isolate the first portion of the channel network from the second portion of the channel network and prevent diffusive transport of particles therebetween. A constriction may be placed in the flow path to reduce the flow rate of the predetermined fluid flowing therethrough. The first and second portions of the channel network are in fluid communication through a junction. The junction intersects the flow path and the constriction is upstream of the junction.

The method may include the additional step of stopping the flow of the predetermined fluid to allow diffusive transport of particles between the first and second portions of the channel network. Alternatively, the flow rate of the predetermined fluid may be reduced to allow particles of a predetermined minimum size to diffuse between the first and second portions of the channel network. A first object may be deposited in the first portion of the channel network and a second object may be deposited in the second portion of the channel network.

In accordance with a further aspect of the present invention, a method is provided of regulating diffusive transport of particles between first and second portions of a channel network in a microfluidic device. The first and second portions of the channel network are in fluid communication. The method includes the steps of depositing a first object in a first portion of the channel network and depositing a second object in the second portion of the channel network. Thereafter, diffusive transport of particles between the first and second portions of the channel network is selectively controlled.

A flow path may be provided in the microfluidic device. The flow path has an input and an output and extends between the first and second portions of the channel network. The diffusive transport is controlled by flowing a predetermined fluid along the flow path at a flow rate. The fluid isolates the first portion of the channel network from the second portion of the channel network and prevents the diffusive transport therebetween. Alternatively, the fluid may flow along the flow path at a predetermined flow rate so as to allow particles of a predetermined minimum size to diffusive between the first and second portions of the channel network. The fluid flowing along the flow path may be stopped to allow diffusive transport of particles between the first and second portions of the channel network.

A constriction may be provided in the flow path. The first and second portions of the channel network are in fluid communication through a junction. The junction intersects the flow path and the constriction is upstream of the junction.

In accordance with a still further aspect of the present invention, a microfluidic device is provided. The microfluidic device includes a body defining an input, an output, a channel network having first and second portions communicating with each other through a junction and a flow path extending from the input to the output through the junction. A flow constriction is provided in the flow path upstream of the junction.

A first introduction port communicating with the first portion of the channel network and a second introduction port communicating with the second portion of the channel network. The first portion of the channel network is in fluid communication with the second portion of the channel network. A first biological object is disposed in the first portion of the channel network. A second biological object is disposed in the second portion of the channel network. A fluid selectively flows along the flow path at a flow rate. The fluid controls diffusion between the first and second biological objects.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an isometric view of a microfluidic device in accordance with the present invention;

FIG. 2a is a cross-sectional view of the microfluidic device of the present invention taken along line 2a-2a of FIG. 1;

FIG. 2b is a cross-sectional view of the microfluidic device of the present invention taken along line 2b-2b of FIG. 1;

FIG. 3 is a schematic, cross-sectional view of the microfluidic device of the present invention taken along line 3-3 of FIG. 2a;

FIG. 4 is a cross-sectional view, similar to FIG. 3, showing an initial stage of diffusive transport between a source region and a destination region of a channel network within the microfluidic device;

FIG. 5 is a cross-sectional view, similar to FIG. 3, showing an advanced stage of diffusive transport between the source region and the destination region of the channel network within the microfluidic device;

FIG. 6 is a cross-sectional view, similar to FIG. 3, showing prevention of the diffusive transport between the source region and the destination region of the channel network with the microfluidic device in accordance the method of the present invention; and

FIG. 7 is a cross-sectional view, similar to FIG. 3, showing termination of the diffusive transport between the source region and the destination region of the channel network with the microfluidic device in accordance with the method of the present invention.

### DETAILED DESCRIPTION OF THE DRAWINGS

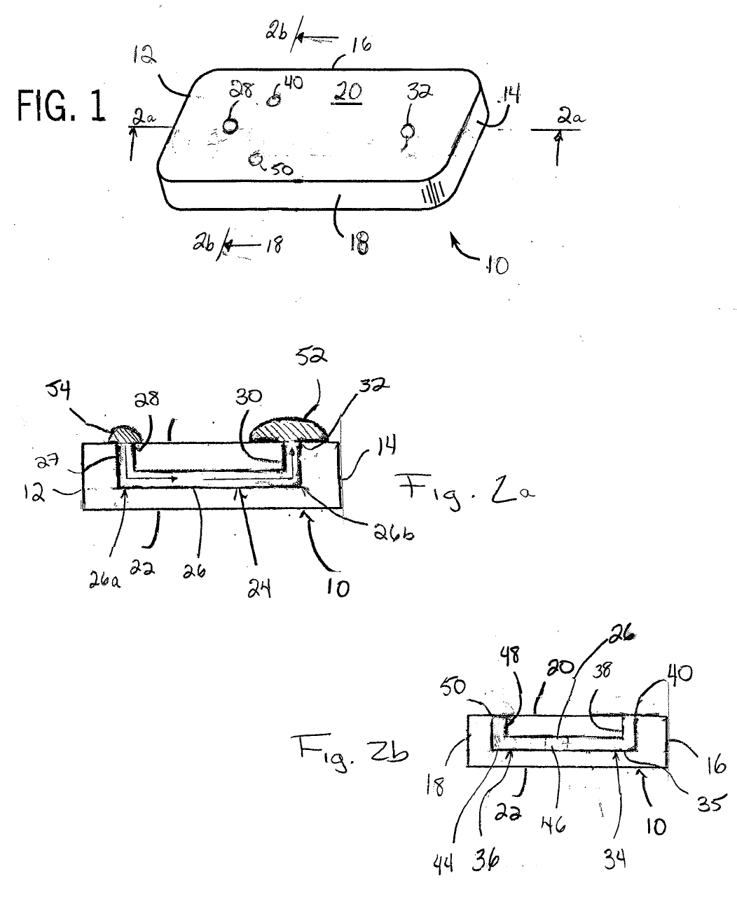

Referring to FIG. 1, a microfluidic device for use in the method of the present invention is generally designated by the reference numeral 10. By way of example, microfluidic device 10 may be fabricated from polydimethylsiloxane (PDMS) and includes first and second ends 12 and 14, respectively, and first and second sides 16 and 18, respectively. In addition, mircofluidic device 10 includes upper and lower surfaces 20 and 22, respectively. While microfluidic device 10 has a generally rectangular configuration in the depicted embodiment, other configurations are contemplated without deviating from the scope of the present invention.

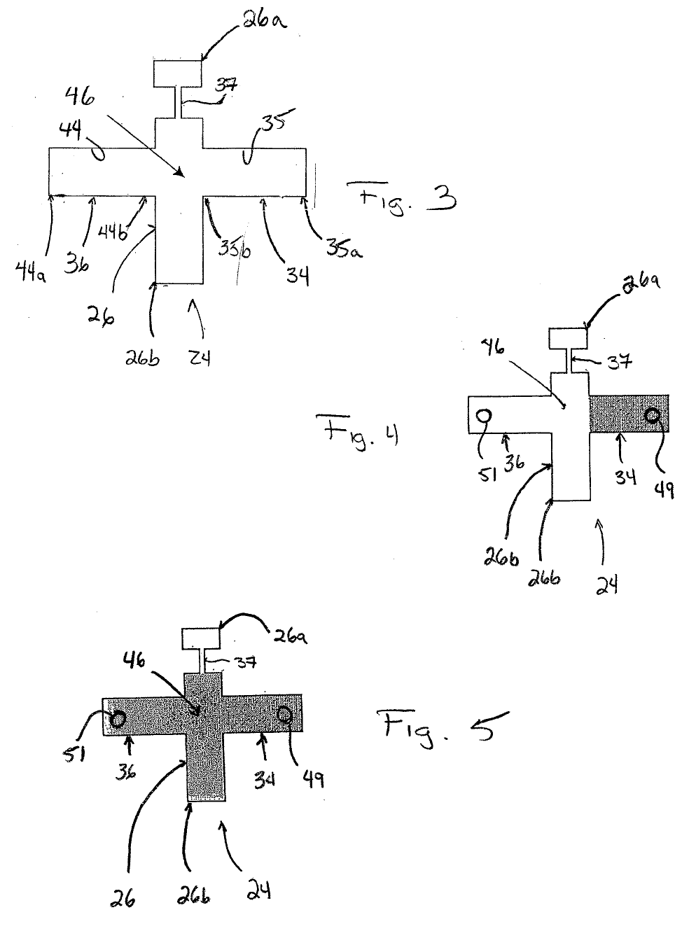

Referring to FIGS. 2a-7, microfluidic device 10 defines channel network 24 extending through the interior thereof. Channel network 24 includes central channel 26 extending along an axis. Central channel 26 has a first end 26a adjacent first end 12 of microfluidic device 10 and a second end 26b adjacent second end 14 of microfluidic device 10. First vertical portion 27 of channel network 24 projects from and communicates with first end 26a of central channel 26. First vertical portion 27 terminates at input port 28 that communicates with upper surface 20 of microfluidic device 10, FIG. 1. Second vertical portion 30 of channel network 24 projects from and communicates with second end 26b of central channel 26. Second vertical portion 30 terminates at output port 32 that also communicates with upper surface 20 of microfluidic device 10, FIG. 1. As best seen in FIGS. 3-7, central channel 26 has a reduced diameter portion 37 adjacent first end 26a thereof, for reasons hereinafter described.

Referring to FIGS. 2b-7, channel network 24 further includes source region 34 and destination region 36. Source region 34 includes a horizontal source channel 35 having a first end 35a adjacent first side 16 of microfluidic device 10 and a second end 35b communicating with central channel 26. First vertical source portion 38 of source region 34 projects from and communicates with first end 35a of source channel 35. First vertical source portion 38 terminates at input port 40 that communicates with upper surface 20 of microfluidic device 10, FIGS. 1 and 2b. Destination region 36 includes a horizontal destination channel 44 having a first end 44a adjacent second side 18 of microfluidic device 10 and a second end 44b communicating with central channel 26. Destination channel 44 is axially aligned with source channel 35 and communicates with source channel 35 though communication portion 46 of central channel 26. First vertical destination portion 48 of destination region 36 projects from and communicates with first end 44a of destination channel 44. First vertical destination portion 48 terminates at input port 50 that communicates with upper surface 20 of microfluidic device 10, FIGS. 1 and 2b.

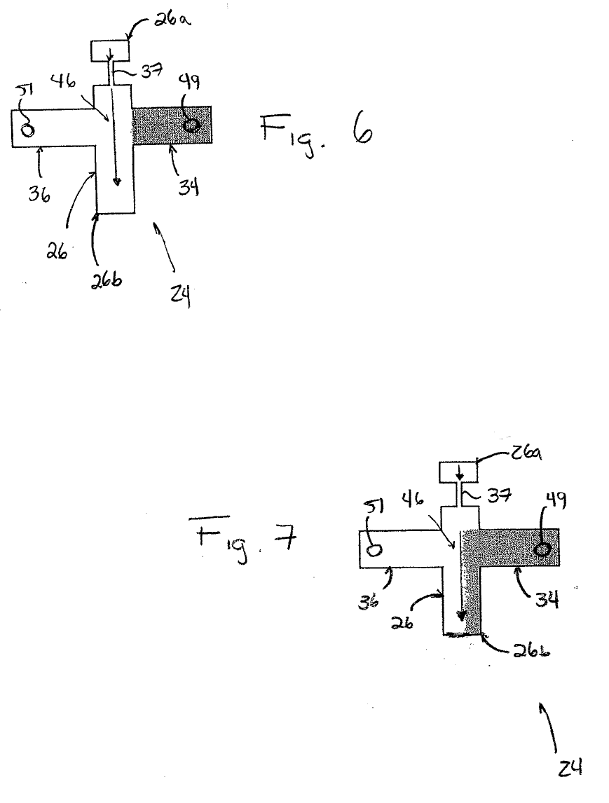

In operation, channel network 24 is filled with a fluid. Thereafter, a user-desired object such as a cell, molecule or the like 49 is introduced into source region 34 though input port 40. Similarly, a user-desired object such as a cell, molecule or the like 51 is introduced into destination region 36 though input port 50. As best seen in FIG. 4, the object in source region 34 of channel network 24 may act as a source of diffusing molecules. Over time, the molecules diffused by the object in source region 34 of channel network 24 enter the destination region 36 through communication portion 46 of central channel 26 and communicate with the object therein, FIG. 5. As a result, signaling between the object in the source region 34 and the object in destination region 36 may be observed for study.

In order to terminate the object to object communication, a large reservoir drop 52 is deposited by a micropipette of robotic micropipetting station over output port 32 of channel network 24, FIG. 2. The radius of reservoir drop 52 is greater than the radius of output port 32 and is of sufficient dimension that the pressure at output port 32 of channel network 24 is essentially zero. A pumping drop 54, of significantly smaller dimension than reservoir drop 52, is deposited on input port 28 of channel network 24. Pumping drop 54 may be hemispherical in shape or may be other shapes. As such, it is contemplated that the shape and the volume of pumping drop 54 be defined by the hydrophobic/hydrophilic patterning of the surface surrounding input port 28 in order to extend the pumping time of the method of the present invention. As heretofore described, microfluidic device 10 is formed from PDMS which has a high hydrophobicity and has a tendency to maintain the hemispherical shapes of pumping drop 54 and reservoir drop 52 on input and output ports 28 and 32, respectively. It is contemplated as being within the scope of the present invention that the fluid in channel network 24, pumping drops 54 and reservoir drop 52 be the same liquid or different liquids.

Because pumping drop 54 has a smaller radius than reservoir drop 52, a larger pressure exists on the input port 28 of channel network. The resulting pressure gradient causes the pumping drop 54 to flow from input port 28 through channel network 24 towards reservoir drop 52 over output port 32 of channel network 24. It can be understood that by sequentially depositing additional pumping drops 54 on input port 28 of channel network 24 by the micropipette of the robotic micropipetting station, the resulting pressure gradient will cause the pumping drops 54 deposited on input port 28 to flow through channel network 24 towards reservoir drop 52 over output port 32 of channel network 24. As a result, fluid flows through central channel 26 of channel network 24 from input port 28 to output port 32. A constriction such as reduced diameter portion 37 of central channel 26 of channel network 24 is provided upstream of communication portion 46 in order to reduce the flow rate of the fluid flowing through central channel 26 of channel network 24 from input port 28 to output port 32.

It can be appreciated that given sufficient fluid flow through central channel 26 of channel network 24, the diffusive transport of molecules from source region 34 into communication portion 46, and hence, into destination region 36 may be terminated, FIG. 6. Alternatively, by reducing the flow rate of the fluid flow through central channel 26 of channel network 24, the fluid flowing through central channel 26 of channel network 24 may be used to capture the molecules diffusing into communication portion 46 and carry such molecules to output port 32 of channel network 24, FIG. 7. Further, it can be appreciated that by slowing the flow rate of the fluid flowing through central channel 26 of channel network 24, molecules of a predetermined size may be able to pass through the fluid flowing through communication portion 46 of channel network 24 into destination region 36.

The flow rate of the fluid flowing through central channel 26 of channel network 24 may be varied by changing the dimensions of central channel 26 and/or the dimensions of reduced diameter portion 37 of central channel 26. Alternatively, the flow rate of the fluid flowing through central channel 26 of channel network 24 may be varied by changing the volume of reservoir drop 52 and/or the volume of pumping drop 54.

Various modes of carrying out the invention are contemplated as being within the scope of the following claims particularly pointing out and distinctly claiming the subject matter, which is regarded as the invention.

We claim:

1. A method of controlling diffusive transport between first and second portions of a channel network in a microfluidic device, the first and second portions of the channel network being in fluid communication, comprising the steps:
providing a flow path in the microfluidic device, the flow path having an input and an output and extending between the first and second portions of the channel network; and
flowing a predetermined fluid along the flow path at a flow rate so as to selectively control diffusive transport of particle between the first and second portions of the channel network.

2. The method of claim 1 wherein the step of flowing the predetermined fluid includes the additional step of increasing the flow rate of the predetermined fluid to predetermined level to isolate the first portion of the channel network from the second portion of the channel network and prevent diffusive transport of particles therebetween.

3. The method of claim 1 further comprising the additional step of placing a constriction in the flow path to reduce the flow rate of the predetermined fluid flowing therethrough.

4. The method of claim 3 wherein:
the first and second portions of the channel network are in fluid communication through a junction;
the junction intersects the flow path; and
the constriction is upstream of the junction.

5. The method of claim 1 further comprising the additional step of stopping the flow of the predetermined fluid to allow diffusive transport of particles between the first and second portions of the channel network.

6. The method of claim 1 further comprising the additional step of reducing the flow rate of the predetermined fluid to allow particles of a predetermined minimum size to diffuse between the first and second portions of the channel network.

7. The method of claim 1 comprising the additional steps of:
depositing a first object in the first portion of the channel network; and
depositing a second object in the second portion of the channel network.

8. A method of regulating diffusive transport of particles between first and second portions of a channel network in a microfluidic device, the first and second portions of the channel network being in fluid communication, comprising the steps:
depositing a first object in a first portion of the channel network;
depositing a second object in the second portion of the channel network; and
selectively controlling diffusive transport of particles between the first and second portions of the channel network.

9. The method of claim 8 comprising the additional step of providing a flow path in the microfluidic device, the flow path having an input and an output and extending between the first and second portions of the channel network.

10. The method of claim 9 wherein the step of controlling diffusive transport includes the step of flowing a predetermined fluid along the flow path at a flow rate so as to isolate the first portion of the channel network from the second portion of the channel network and prevent the diffusive transport therebetween.

11. The method of claim 9 wherein the step of controlling diffusive transport includes the step of flowing a predetermined fluid along the flow path at a predetermined flow rate so as to allow particles of a predetermined minimum size to diffusive between the first and second portions of the channel network.

12. The method of claim 9 further comprising the additional step of placing a constriction in the flow path.

13. The method of claim 12 wherein:
the first and second portions of the channel network are in fluid communication through a junction;
the junction intersects the flow path; and
the constriction is upstream of the junction.

14. The method of claim 9 wherein the step of selectively controlling diffusive transport of particles includes the step of flowing a predetermined fluid along the flow path.

15. The method of claim 14 further comprising the additional step of stopping the flow of the predetermined fluid to allow diffusive transport of particles between the first and second portions of the channel network.

16. A microfluidic device, comprising:
a body defining an input, an output, a channel network having first and second portions communicating with each other through a junction and a flow path extending from the input to the output through the junction; and
a flow constriction in the flow path upstream of the junction.

17. The microfluidic device of claim 16 further comprising a first introduction port communicating with the first portion of the channel network.

18. The microfluidic device of claim 17 further comprising a second introduction port communicating with the second portion of the channel network.

19. The microfluidic device of claim 16 wherein the first portion of the channel network is in fluid communication with the second portion of the channel network.

20. The microfluidic device of claim 16 further comprising a first biological object disposed in the first portion of the channel network, a second biological object disposed in the second portion of the channel network, and fluid selectively flowing along the flow path at a flow rate, the fluid controlling diffusion between the first and second biological objects.
